(19) United States
(12) Patent Application Publication
(10) Pub. No.: US 2025/0134348 A1
(43) Pub. Date: May 1, 2025

(54) IMAGE PROCESSING DEVICE, IMAGE PROCESSING METHOD, AND STORAGE MEDIUM

(71) Applicant: OLYMPUS MEDICAL SYSTEMS CORP., Tokyo (JP)

(72) Inventor: Hiroshi TANAKA, Tokyo (JP)

(73) Assignee: OLYMPUS MEDICAL SYSTEMS CORP., Tokyo (JP)

(21) Appl. No.: 18/926,779

(22) Filed: Oct. 25, 2024

Related U.S. Application Data
(60) Provisional application No. 63/546,558, filed on Oct. 31, 2023.

(51) Int. Cl. A61B 1/00; G06T 7/00; G06T 7/20; G06T 17/00; G06V 20/50

(52) U.S. Cl. CPC A61B 1/000096; G06T 7/0012; G06T 7/20; G06T 17/00; G06V 20/50; G06T 2207/10068; G06T 2207/30096; G06T 2207/30168; G06T 2210/41; G06V 2201/034

(57) ABSTRACT

Provided is an image processing device including one or more processors including hardware, wherein the processor is configured to: identify a status of observation of an observation target based on an image captured by an endoscope; determine, based on the status of observation, a processing mode concerning detection of an unobserved region that has not been observed by the endoscope. The processor is further configured to perform generation of a three-dimensional model of the observation target from the image and detection of the unobserved region according to the determined processing mode.

### CROSS-REFERENCE TO RELATED APPLICATIONS

This application claims the benefit of U.S. Provisional Application No. 63/546,558, filed Oct. 31, 2023, which is hereby incorporated by reference herein in its entirety.

### TECHNICAL FIELD

The present disclosure relates to an image processing device, an image processing method, and a storage medium.

### BACKGROUND

In the related art, there is a known technology in which a three-dimensional (3D) model of an observation target is reconstructed in real time from images acquired by an endoscope. Unobserved regions that have not been observed by the endoscope are not reconstructed in the three-dimensional model. In Patent Literature 1, the unobserved regions that have not been reconstructed are detected, and a warning is issued to an endoscopist.

### SUMMARY

An aspect of the present disclosure is an image processing device, the image processing device comprising: one or more processors comprising hardware, wherein the one or more processors being configured to: identify a status of observation of an observation target based on an image captured by an endoscope; determine, based on the status of observation, a processing mode concerning detection of an unobserved region that has not been observed by the endoscope; and perform generation of a three-dimensional model of the observation target from the image and detection of the unobserved region according to the determined processing mode.

Another aspect of the present disclosure is An image processing method, the image processing method comprising: identifying a status of observation of an observation target based on an image captured by an endoscope; determining, based on the status of observation, a processing mode concerning detection of an unobserved region that has not been observed by the endoscope, generating a three-dimensional model of the observation target from the image and detecting of the unobserved region are performed according to the processing mode.

Another aspect of the present disclosure is a non-transitory computer-readable storage medium that stores an image processing program, wherein the image processing program causes the computer to execute: identifying a status of observation of an observation target based on an image captured by an endoscope; determining, based on the status of observation, a processing mode concerning detection of an unobserved region that has not been observed by the endoscope; and generating a three-dimensional model of the observation target from the image and detecting of the unobserved region according to the processing mode.

### BRIEF DESCRIPTION OF DRAWINGS

FIG. 1 is a block diagram showing the configuration of an image processing device and an endoscope system according to a first embodiment.

FIG. 2 is a diagram for explaining an unobserved region in a three-dimensional model.

FIG. 3A is a diagram showing a display example in an execution mode.

FIG. 3B is a diagram showing a display example in a stop mode.

FIG. 3C is a diagram showing another display example in the stop mode.

FIG. 4A is a diagram showing another display example in the execution mode.

FIG. 4B is a diagram showing another display example in the execution mode.

FIG. 5A is a diagram showing an example of a report.

FIG. 5B is a diagram showing another example of the report.

FIG. 6A is a flowchart of an image processing method according to the first embodiment of the present disclosure.

FIG. 6B is a flowchart for explaining step S2 and step S3.

FIG. 7A is a diagram showing an example of a 3D model displayed on a display device in a second embodiment.

FIG. 7B is a diagram showing another example of the 3D model displayed on the display device in the second embodiment.

FIG. 8 is a flowchart for explaining step S2 and step S3 in an image processing method according to the second embodiment.

FIG. 9 is a flowchart for explaining step S2 and step S3 in an image processing method according to a third embodiment.

### DESCRIPTION OF EMBODIMENTS

In endoscopic examinations, it is necessary to inspect all organs to be examined for the presence or absence of diseases to prevent oversight during examinations, and to perform efficient examinations that reduce the burden on the patients (subjects) being examined.

Therefore, while technology has been developed to assist doctors, and it is possible to perform efficient examinations by judging images so that they are not missed, if the technology becomes an aid that puts more time and effort than necessary on the examination, it will deviate from the original purpose. Therefore, an example for the purpose of technical improvement that optimizes the detection performance according to the part such as in the organ and makes it sharp, and balances speed and oversight will be described. In addition, the data that quantifies oversights can be used for standardization, skill level, and test quality, and is valuable for quantifying skills in the management of medical institutions. It is also effective in determining the priority observation site in the secondary inspection, which leads to the judgment of the period until the next inspection.

### First Embodiment

An image processing device, an image processing method, an image processing program, and a storage medium according to a first embodiment of the present disclosure will be described with reference to the drawings.

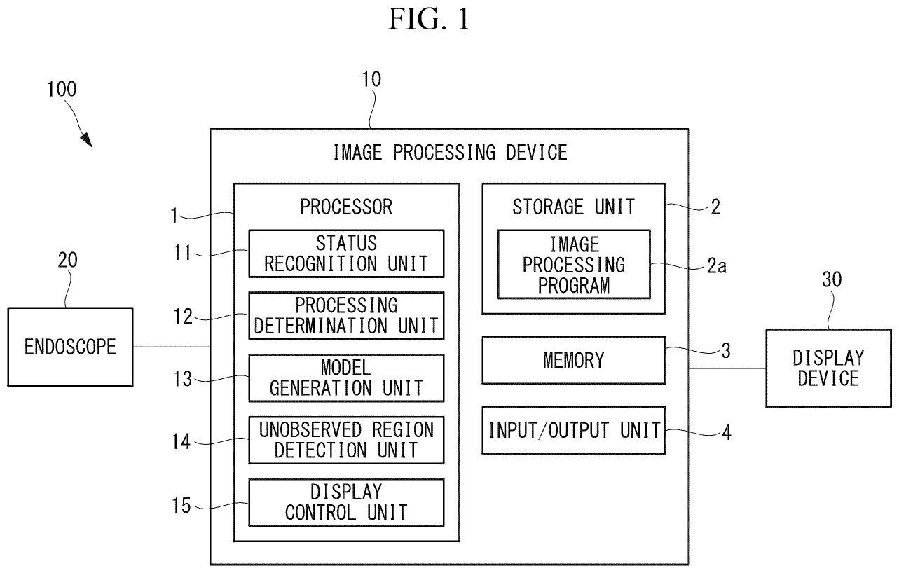

FIG. 1 shows an endoscope system 100 to which an image processing device 10 according to this embodiment is applied. The endoscope system 100 includes an endoscope 20, the image processing device 10 that processes images captured by the endoscope 20, and a display device 30.

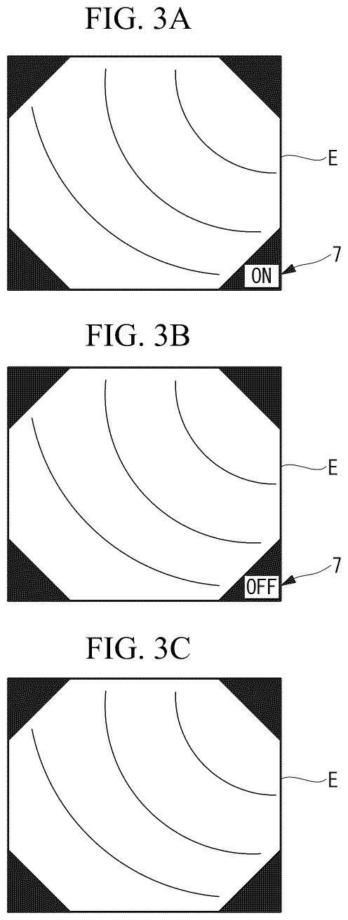

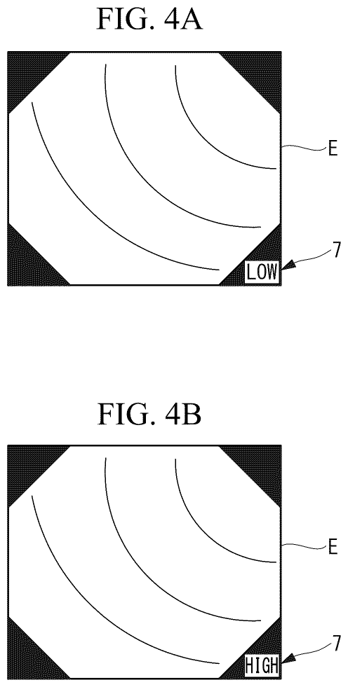

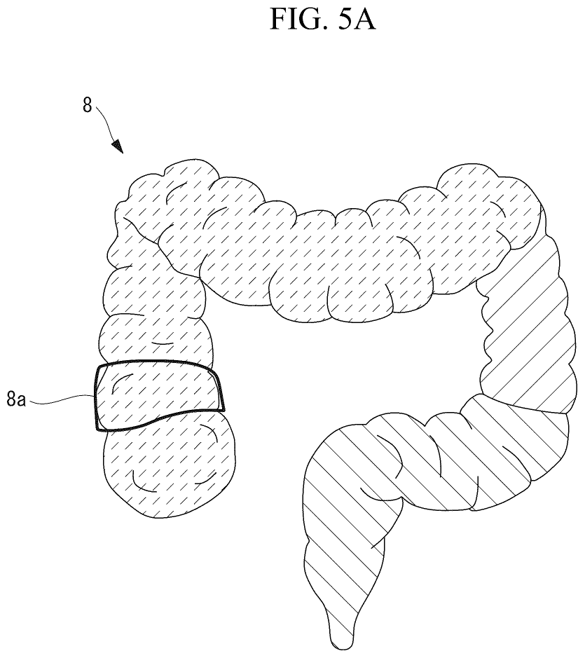

The endoscope 20 is, for example, a colonoscope. The endoscope 20 may be another type of endoscope. The display device 30 is a publicly known display, such as a liquid crystal display. An image E captured by the endoscope 20 is input to the display device 30 through the image processing device 10 and is displayed on the display device 30 (see FIGS. 3A to 4B).

The image processing device 10 has a processor 1 such as a central processing unit, a storage unit 2, a memory 3, and an input/output unit 4. For example, the image processing device 10 is composed of an arbitrary computer such as a personal computer.

The storage unit 2 is a non-transitory computer-readable storage medium and is, for example, a publicly known magnetic disk, optical disk, flash memory, or the like. The storage unit 2 stores an image processing program 2a for causing the processor 1 to execute an image processing method, which will be described later.

The memory 3 is composed of a volatile storage device, such as a random access memory (RAM), and is used as a work area of the processor 1.

The input/output unit 4 has a publicly known input/output interface, and the image processing device 10 is connected to the endoscope 20 and the display device 30 via the input/output unit 4.

The processor 1 has, as functional units, a status recognition unit 11, a processing determination unit 12, a model generation unit 13, an unobserved region detection unit 14, and a display control unit 15.

The status recognition unit 11 acquires the image E input from the endoscope 20 to the image processing device 10 and recognizes, on the basis of the image E, the current status of observation of an observation target, performed by a user such as a doctor. In the endoscopy, the observation target is an organ (for example, the large intestine) to be examined, and the status of observation is the status of examination of a mucous membrane to be examined by the user. Hereinafter, the status of observation is also referred to as the status of examination.

Specifically, the status recognition unit 11 recognizes an observation site in the image E and classifies the recognized observation site as either a detection target or a non-detection target. For example, the status recognition unit 11 is provided with artificial intelligence (AI) that has learned images of individual sites of the large intestine, which is the observation target, and recognizes an observation site in an image by means of the AI. By doing so, the status of examination is recognized as the user observing a detection target, or otherwise.

The detection target is a site where detection of an unobserved region that has not been observed by the endoscope 20 should be executed and is, for example, a site to be examined, which a doctor examines for the presence or absence of a lesion site. The non-detection target is a site other than the detection target.

For example, in general colonoscopy, the user inserts the endoscope 20 from the anus to the cecum, and subsequently examines individual sites of the large intestine while removing the endoscope 20 from the cecum to the anus. Therefore, in the colonoscopy, it is unnecessary to detect unobserved regions from an image E captured outside the body and an image E captured during insertion of the endoscope 20, and the detection target is each site of the large intestine from the cecum to the anus during removal of the endoscope 20. The status recognition unit 11 classifies, for example, observation sites before being recognized to be the cecum as non-detection targets, and classifies individual sites of the large intestine, after the observation sites are recognized to be the cecum, as detection targets. This site detection may be determined by anatomical features from the image obtained by the endoscope, or by the shape obtained by 3D reconstruction, or by the elapsed time since the start of the endoscopy, the amount of endoscope insertion, the direction (angle) of the tip, and the like.

The detection targets include high-risk sites and low-risk sites. The high-risk site is a site where there is a high risk of overlooking a lesion site, and examples thereof include a common site for lesions and a site with many folds. The low-risk site is an observation site where there is a low risk of overlooking a lesion site, and examples thereof include a site having a low risk of lesions occurring and a flat site with fewer folds. In order to detect the presence or absence of such lesions, for example, an image of a target part with a lesion selected from a pre-prepared endoscopic image or an image of a target part without a lesion is annotated with the presence or absence of a lesion and the like, and an inference model learned as teacher data is created. In that case, if the reliability and similarity required are increased, the detection threshold value is increased, and the detection sensitivity is reduced. In this case, it is difficult to find lesions other than the one expected. Conversely, if the reliability and similarity required at that time are lowered, the detection threshold value is lowered, and the detection sensitivity is increased. In this case, it is easy to find lesions other than the expected lesions, but false positives increase, and it is necessary to design with this balance in mind. It is better to increase the sensitivity to the place where it is difficult for the doctor to find it (depending on the part), and even if it is a false detection, the doctor's awareness can be directed there so that it is difficult to miss it. As an example, in areas with high/large folds (since it is determined anatomically, it is good to record the difficulty and ease of overlooking each part in a database, etc.), there is a high possibility of oversight, so increase sensitivity. In the opposite area, the sensitivity is reduced. That is, the image processing apparatus here detects the features of the observation target site from the obtained continuous captured images, and according to the characteristics of this site, the image processing mode related to the detection of the area that is likely to be unobserved is determined. From the obtained image, the position of the target site and the complexity of the structure itself may be determined, or as a result of the site determination, the characteristics of the site (especially ease of inspection) may be searched from the database and determined.

The processing determination unit 12 determines processing modes concerning processing of unobserved regions on the basis of the result of classification of the observation sites. The processing modes include an execution mode for executing detection of unobserved regions and a stop mode for stopping detection of unobserved regions. The processing mode may include the method of determining reliability and similarity described above, and the ON/OFF of the detection function itself.

Furthermore, the execution mode includes a plurality of modes in which the detection sensitivities for unobserved regions are different from each other. In this embodiment, the plurality of modes are a low-sensitivity mode in which unobserved regions are detected with a prescribed sensitivity and a high-sensitivity mode in which unobserved regions are detected with a sensitivity higher than the sensitivity in the low-sensitivity mode. The sensitivity is changed, for example, according to a definition size defined as an unobserved region. As the definition size becomes smaller, the sensitivity is set to be higher, and the larger the definition size, the lower the sensitivity. The sensitivity may be changed according to a definition size presented as an unobserved region. As an example of a plurality of modes, not only the size of the object but also the tolerance of differences in color changes, image processing such as sensitization processing of the imaging result or high-resolution processing, and special light may be used in combination. In other words, in order to increase the sensitivity, it is necessary to call attention when there is even a slight possibility, so even if various image processing, special light, etc. are used together, it is tried to find the lesion, but it takes time and processing time, and there is a burden on the doctor and patient, and the load and power consumption of the display control unit and the computer circuit increase. The lower the sensitivity, the less likely that the problem will occur. In the previous example, in the area with high/many folds, it is necessary to find lesions of various sizes in the depth direction, etc., and there is a high possibility of oversight, so the sensitivity is increased so that various sizes of lesions can be accommodated. Other parameters related to sensitivity include frame rate, number of pixels, detection area, and exposure sensitization.

The processing determination unit 12 selects the high-sensitivity mode when an observation site is classified as a high-risk site, selects the low-sensitivity mode when an observation site is classified as a low-risk site, and selects the stop mode when an observation site is classified as a non-detection target.

The execution mode may further include a plurality of modes in which processing parameters other than the detection sensitivities are different from each other. The reason for changing the detection sensitivity here is that, as described above, even if there is a false positive, the doctor's awareness may be raised, and even if the doctor's awareness is sufficiently high, if there is a false positive, it may be troublesome.

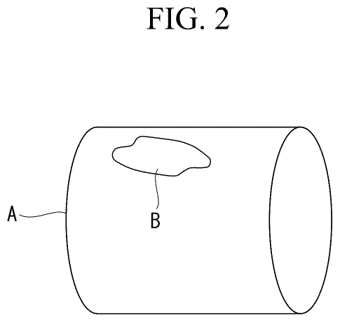

In a case in which the processing determination unit 12 determines the processing mode to be the execution mode (in other words, the low-sensitivity mode or the high-sensitivity mode), the model generation unit 13 generates a three-dimensional (3D) model A of the observation target from the image E (see FIG. 2). For the generation of the 3D model A, a publicly known three-dimensional reconstruction technology, such as visual simultaneous localization and mapping (SLAM), is used.

The model generation unit 13 may generate a 3D model A of only a specific region of the observation target. For example, the model generation unit 13 may detect a region of folds in the image E by means of image recognition utilizing deep learning and generate a 3D model A of only the region of folds. In addition to the area of the object to be observed obtained in the 3D model reconstructed from the 2D endoscopic image, the area of the object to be observed may be calculated from the image acquired and created by CT or MRI.

In the case in which the processing determination unit 12 determines the processing mode to be the execution mode, the unobserved region detection unit 14 detects an unobserved region B in the 3D model A. As shown in FIG. 2, an unobserved region of the observation target, which has not been observed by the endoscope 20, forms a missing region B in which the shape of the observation target is not restored in the 3D model A. The unobserved region detection unit 14 detects the missing region B as an unobserved region by using the definition size corresponding to the determined mode. In other words, the unobserved region detection unit 14 detects the unobserved region B according to a standard definition size in a case of the low-sensitivity mode, and detects the unobserved region B according to a definition size smaller than the standard definition size in a case of the high-sensitivity mode. The definition size corresponds to, for example, and is the area of the target portion that detects the feature when detecting a specific image feature, or the size of the object occupied by the object in the screen. Further, the unobserved region may be determined when there is a frame that does not apply when an endoscopic image frame corresponding to each part of the 3D model of the luminal portion obtained in advance is applied. The common parts of the image features of the plurality of image frames taken continuously at the time of inspection are glued together, and the frames that are not connected when the images are connected in a panoramic manner are used as unobserved areas, or related frames may be connected and the gaps created may be used as unobserved areas.

As shown in FIGS. 3A to 5B, the display control unit 15 generates displays 7, 8 concerning the detection of the unobserved region B and outputs the displays 7, 8 to the display device 30. The display control unit 15 may generate and output one or both of the displays 7, 8.

FIGS. 3A to 4B each show an example of the display 7 that the display control unit 15 generates in real time during the endoscopy. The display 7 indicates whether the detection of the unobserved region B by the unobserved region detection unit 14 is being executed or stopped. The display control unit 15 generates the display 7 on the basis of the processing mode determined by the processing determination unit 12, and outputs the display 7 to the display device 30 together with the image E. When the processing mode is changed, the display control unit 15 also changes the display 7 in real time.

In the examples in FIGS. 3A to 3C, a color display 7 indicating “ON” is displayed in the execution mode (see FIG. 3A). In the stop mode, a gray-out display 7 indicating “OFF” is displayed (see FIG. 3B) or the display 7 disappears (see FIG. 3C).

The display 7 may be changed according to the detection sensitivity for the unobserved region B. For example, the color or text of the display 7 may be different between the high-sensitivity mode and the low-sensitivity mode. In the examples in FIGS. 4A and 4B, a display 7 indicating “low” is displayed in the low-sensitivity mode and a display 7 indicating “high” is displayed in the high-sensitivity mode.

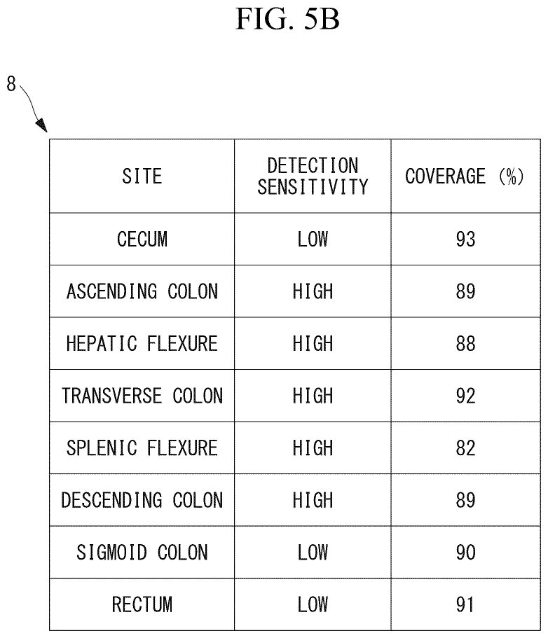

FIGS. 5A and 5B each show an example of the display 8 that the display control unit 15 generates after the observation of the observation target by the endoscope 20 is completed. The display 8 is a report of the observation result. For example, in the case of endoscopy, the report 8 may include information on the observation coverage for individual sites of an organ to be examined, and may include information on the detection sensitivity for the unobserved region B in each site. The observation coverage is calculated on the basis of the surface area of the 3D model A of the organ and the area of the unobserved region B. The surface area of the 3D model A of the organ may be calculated by estimating the lumen center of the 3D model, creating a developed view, and calculating the surface area with the created developed view. Further, the area of the target portion of each image frame may be determined from the angle of view of the image obtained by the endoscope and the distance to the observation target portion, and it may be integrated. Further, the endoscope may have the function of distance measurement and distance inference, and a general observation distance or the like may be used as a multiplier. In this way, the observation coverage rate can be calculated by dividing the area of the observed region obtained from the image at the time of observation by the area of the object area to be observed. Further, the coverage rate cannot be calculated only by the area, and may be based on the number of folds inside the specific lumen. In other words, the ratio of the number of observed folds to the total number of folds can also be said to be the observation coverage ratio.

The report 8 in FIG. 5A includes a schema diagram of the large intestine, and each site of the large intestine is displayed in a form corresponding to the observation coverage. For example, a site with a high observation coverage and a site with a low observation coverage are displayed in different colors. The sensitivity in each site may be expressed by, for example, the color, thickness, type, or the like of a line 8a surrounding each site.

The report 8 in FIG. 5B is a table showing the sensitivity and the observation coverage for the individual sites.

Next, an image processing method executed by the image processing device 10 will be described.

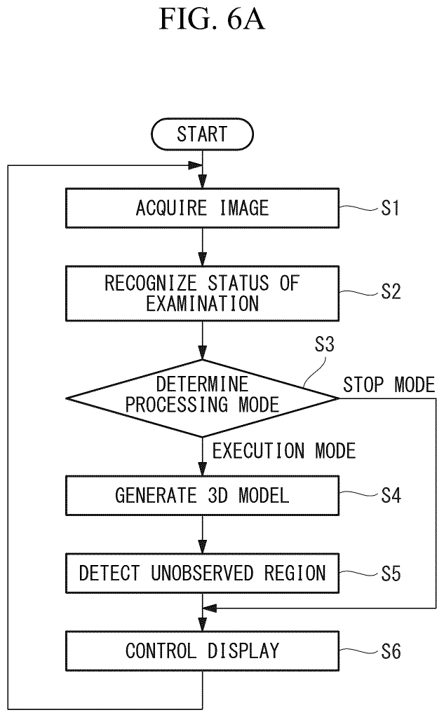

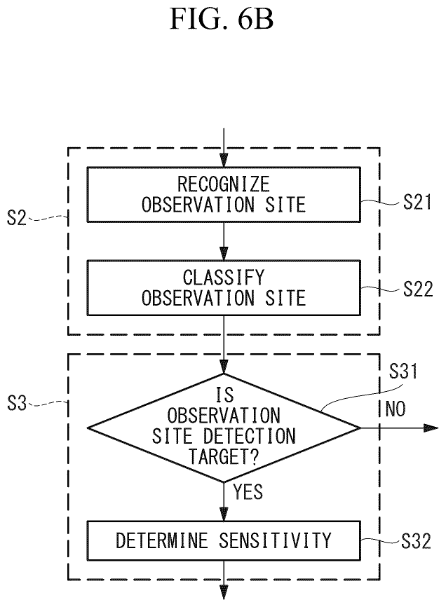

As shown in FIG. 6A, the image processing method according to this embodiment includes: step S1 of acquiring an image E captured by the endoscope 20; step S2 of recognizing the status of examination on the basis of the image E; step S3 of determining a processing mode on the basis of the status of examination; step S4 of generating a 3D model A of the observation target from the image E; step S5 of detecting an unobserved region B on the basis of the 3D model A; and step S6 of generating and displaying a display 7 concerning the detection of the unobserved region B. An example of an organ site is also illustrated in FIG. 5B, but it may be classified into a finer part, and when the coverage rate is low in that range, such as S6 in FIG. 6A described later, the site may be prompted. As for the coverage of the unobserved region, the same figure (not limited to the large intestine) as the Shema diagram of the large intestine in FIG. 5A is displayed side by side on the endoscopic image, or at the same time at the time of endoscopy on another display or the like. Further, when the coverage rate is good, a color, text, or symbol indicating the completion of the part may be displayed. Similarly, the report 8 of FIG. 5B may be displayed side by side on the endoscope image or at the same time at the time of endoscopy on another display or the like, and when the coverage rate is low on the table of the report, a user interface may be adopted that flashes the part. Further, when the coverage rate is good, a color, text, or symbol indicating the completion of the part may be displayed. Further, the completed report may be used for the next inspection.

The status recognition unit 11 sequentially acquires images E sequentially input from the endoscope 20 to the image processing device 10 (step S1), and recognizes the current status of examination on the basis of the images E (step S2).

Specifically, as shown in FIG. 6B, the status recognition unit 11 recognizes an observation site in the image E (step S21) in step S2. Next, the status recognition unit 11 classifies the observation site as a detection target or a non-detection target, and in the case in which the observation site is a detection target, the observation site is further classified as a low-risk site or a high-risk site (step S22). By doing so, the status of examination is recognized as a detection target being observed, or otherwise.

Next, the processing determination unit 12 determines a processing mode on the basis of the status of examination (step S3).

Specifically, in the case in which the observation site is a detection target (“YES” in step S31), the processing determination unit 12 subsequently determines the sensitivity in the execution mode (step S32). Specifically, in the case of the low-risk site, the processing determination unit 12 determines the sensitivity to be a prescribed sensitivity, in other words, determines the processing mode to be the low-sensitivity mode. Meanwhile, in the case of the high-risk site, the processing determination unit 12 determines the sensitivity to be a high sensitivity, in other words, determines the processing mode to be the high-sensitivity mode.

In the case in which the observation site is a non-detection target, the processing determination unit 12 determines the processing mode to be the stop mode (“NO” in step S31).

In the case in which the processing mode is determined to be the execution mode (high-sensitivity mode or low-sensitivity mode) (“execution mode” in step S3), the model generation unit 13 generates a 3D model A from the image E (step S4), and the unobserved region detection unit 14 subsequently detects an unobserved region B from the 3D model A in the mode determined in step S32 (step S5).

Meanwhile, in the case in which the processing mode is determined to be the stop mode (“stop mode” in step S3), steps S4 and S5 are not executed and step S6 is executed next.

Next, the display control unit 15 generates a display 7 on the basis of the processing mode determined in step S3, and outputs the display 7 to the display device 30 together with the image E (step S6). The display control unit 15 may generate and output a report 8 after completion of the endoscopy.

As described above, with this embodiment, the current status of examination is recognized on the basis of the images E during the endoscopy, and execution and stopping of the detection of unobserved regions are automatically switched between according to the status of examination. For example, the detection of unobserved regions is executed when a doctor is observing the mucous membrane in the individual sites of the large intestine while removing the endoscope 20, and otherwise, the detection of unobserved regions is automatically stopped. With this configuration, it is possible to prevent more unobserved regions than necessary from being detected for a user such as a doctor.

In addition, with this embodiment, it is possible to prevent the unobserved-region detection results from being excessively presented to the user during or after the endoscopy. For example, in colonoscopy, information concerning unobserved regions before and during the insertion of the endoscope 20 into the large intestine is unnecessary for the user. It is possible to generate a report 8 that does not include such unnecessary information.

In addition, with this embodiment, the detection sensitivity for unobserved regions is changed according to the observation sites in the images during the endoscopy, and thus, the sensitivity is set to be high in sites where unobserved regions should be finely detected or presented. With this configuration, it is possible to more reliably detect and prevent oversight of a high-risk site.

### Second Embodiment

Next, an image processing device, an image processing method, an image processing program, and a storage medium according to a second embodiment of the present disclosure will be described.

This embodiment differs from the first embodiment in the method of recognizing the status of examination and the method of determining a processing mode. In this embodiment, a configuration different from that of the first embodiment will be described, and parts having the same configuration as those in the first embodiment will be assigned the same reference signs, and descriptions thereof will be omitted.

As with the first embodiment, the image processing device 10 according to this embodiment is applied to the endoscope system 100 and includes the processor 1, the storage unit 2, the memory 3, and the input/output unit 4.

The processor 1 has, as functional units, the status recognition unit 11, the processing determination unit 12, the model generation unit 13, the unobserved region detection unit 14, and the display control unit 15.

The status recognition unit 11 recognizes the current status of examination on the basis of the image E. Specifically, in this embodiment, the status recognition unit 11 recognizes identification and treatment by employing at least one of first to sixth methods mentioned below, and by doing so, the current status of examination is recognized as the identification or treatment being performed, or otherwise.

In the first method, the status recognition unit 11 recognizes identification or treatment on the basis of a lesion site in the image E. During the identification or treatment, a lesion site to be identified or treated is present in the image E. For example, the status recognition unit 11 recognizes the lesion site in the image E by means of the AI that has learned images of lesion sites.

In the second method, the status recognition unit 11 recognizes treatment on the basis of a treatment tool in the image E. During the treatment, a treatment tool for treating a lesion site is present in the image E. For example, the status recognition unit 11 recognizes the treatment tool in the image E by means of the AI that has learned images of treatment tools.

In the third method, the status recognition unit 11 recognizes identification on the basis of a staining solution in the image E. When identifying a lesion site, there is a case in which a staining solution is sprayed onto the mucous membrane. For example, the status recognition unit 11 recognizes the staining solution by means of the AI that has learned images of the mucous membrane onto which the staining solution is sprayed.

In the fourth method, the status recognition unit 11 recognizes identification or treatment on the basis of special light in the image E. The special light is light of a specific color and is, for example, blue and green light for narrow band imaging (NBI). In the identification or treatment, there is a case in which observation using the special light, such as NBI, is performed. For example, the status recognition unit 11 recognizes the special light by means of the AI that has learned images of the mucous membrane illuminated with the special light (for example, NBI images).

In the fifth method, the status recognition unit 11 recognizes treatment on the basis of operation of a treatment tool. The treatment tool during the treatment performs a specific operation. For example, the status recognition unit 11 recognizes the operation of the treatment tool during the treatment by means of the AI that has learned operations of the treatment tool during the treatment, such as excision of a lesion site and hemostasis.

In the sixth method, the status recognition unit 11 recognizes identification or treatment on the basis of movement of the observation site in the image E. In general, during the identification or treatment, the same region of the mucous membrane continues to be observed for a certain period of time. For example, the status recognition unit 11 recognizes the identification or treatment on the basis of a state in which the observation target, such as the mucous membrane, in the image E does not move over a certain period of time.

The processing determination unit 12 determines a processing mode according to the status of examination recognized by the status recognition unit 11.

For example, in a case in which neither identification nor treatment is recognized, the processing determination unit 12 determines the processing mode to be the execution mode.

In addition, in a case in which at least one of identification or treatment is recognized, the processing determination unit 12 determines the processing mode to be the execution mode or the stop mode on the basis of preset conditions. The conditions are set in the image processing device 10 by the user before the endoscopy is executed. The user can set whether or not to execute the detection of unobserved regions in each of identification and treatment, for example, by using a user interface (not shown).

In the case in which the processing mode is determined to be the execution mode, the processing determination unit 12 may further determine, on the basis of the status of examination, respective processing algorithms for the generation of a 3D model A and the detection of an unobserved region.

A first example of the processing algorithms is learning models respectively used in the detection of an unobserved region and the generation of a 3D model A. By using learning models corresponding to the status of examination, it is possible to enhance the precision in the generation of a 3D model A and the precision in the detection of an unobserved region.

In one example, the processing determination unit 12 determines, on the basis of the recognition result for the status of examination, a learning model to be used by the AI of the model generation unit 13 in the generation of a 3D model A.

For example, as learning models for the generation of a 3D model A, a stain-solution learning model and an NBI learning model are stored in the storage unit 2 in addition to a normal learning model. The stain-solution learning model is created by means of learning using images of the mucous membrane onto which a staining solution is sprayed. The NBI learning model is created by means of learning using NBI images. In a case in which the staining solution or the NBI image is recognized, the processing determination unit 12 determines the learning model for the generation of a 3D model A to be the stain-solution or NBI learning model.

In another example, the processing determination unit 12 determines, on the basis of the recognition result for the status of examination, a learning model to be used by the AI of the unobserved region detection unit 14 in the detection of an unobserved region.

For example, as learning models for the detection of an unobserved region, a stain-solution learning model and an NBI learning model are stored in the storage unit 2 in addition to a normal learning model. In a case in which the staining solution or the NBI image is recognized, the processing determination unit 12 determines the learning model for the detection of an unobserved region to be the stain-solution or NBI learning model.

A second example of the processing algorithms is the detection range or the detection granularity for an unobserved region. In one example, in a case in which identification is recognized, the processing determination unit 12 limits the detection range for an unobserved region to a peripheral region of a lesion site being identified.

In the case in which the processing mode is determined to be the execution mode, the model generation unit 13 generates a 3D model A, and the unobserved region detection unit 14 detects an unobserved region B.

In the case in which the processing mode is determined to be the stop mode, the model generation unit 13 stops the generation of a 3D model A, and the unobserved region detection unit 14 stops the detection of an unobserved region B.

During the execution of the detection of an unobserved region B, the display control unit 15 causes the 3D model A to be output to the display device 30 and displayed thereon in real time, thereby notifying the user of the unobserved region B. The display control unit 15 may cause the image E to be displayed on a main screen and may cause the 3D model A to be displayed on a sub-screen. The display control unit 15 may cause the 3D model A to be displayed on a sub-monitor different from the display device 30.

During the identification or treatment, the display control unit 15 may cause the 3D model A to be displayed on the display device 30, only in an area surrounding a lesion site being identified or treated.

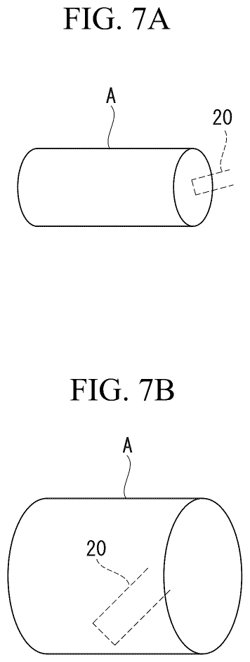

FIGS. 7A and 7B show examples of the 3D model A displayed on the display device 30. In a normal operation, the display control unit 15 may cause the 3D model A around the distal end of the endoscope 20 to be displayed (see FIG. 7A), and when the identification or treatment is performed, the display control unit 15 may cause the 3D model A to be displayed in a manner in which a region to be identified or treated is enlarged (see FIG. 7B).

While the detection of an unobserved region B is stopped, the display control unit 15 need not generate and output a display concerning the unobserved region B. Alternatively, the display control unit 15 may cause information (for example, position information) of the unobserved region B to be displayed, the unobserved region B being detected before the identification or treatment.

Next, an image processing method executed by the image processing device 10 will be described.

As with the first embodiment, the image processing method according to this embodiment includes steps S1 to S6.

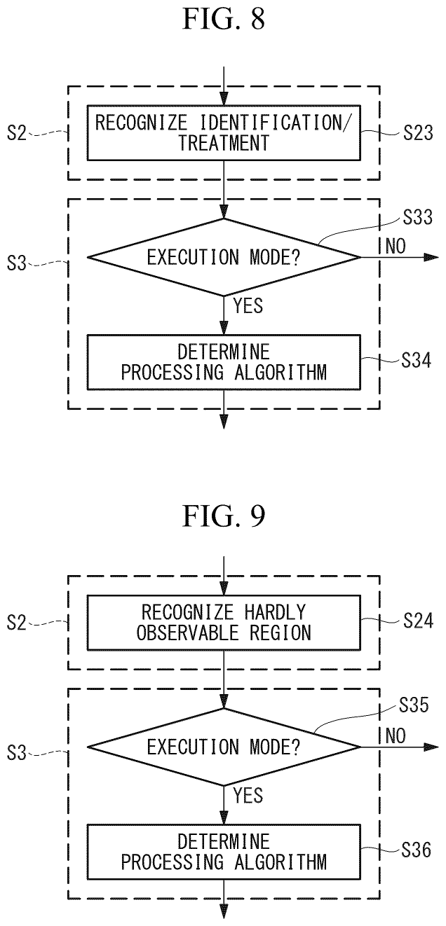

FIG. 8 shows step S2 and step S3 in this embodiment.

In step S2, the status recognition unit 11 recognizes at least one of a lesion site, a treatment tool, a staining solution, an operation of a treatment tool, or special light in the image E, or movement of the observation site, in the image E, and by doing so, the current status of examination is recognized as the identification or treatment being performed, or otherwise (step S23).

Next, in step S3, in a case in which neither of identification and treatment is recognized, the processing determination unit 12 determines the processing mode to be the execution mode (“YES” in step S33), and in a case in which identification or treatment is recognized, the processing determination unit 12 determines the processing mode to be the execution mode or the stop mode on the basis of preset conditions (“YES” or “NO” in step S33).

In the case in which the processing mode is determined to be the execution mode (“YES” in step S33), the processing determination unit 12 subsequently determines processing algorithms (step S34). In this case, steps S4, S5, and S6 are subsequently executed.

Meanwhile, in the case in which the processing mode is determined to be the stop mode (“NO” in step S33), steps S4 and S5 are not executed and step S6 is executed next.

In step S6, the display control unit 15 causes, for example, a 3D model A to be displayed on the display device 30. The user can recognize that there is an unobserved region that has not yet been observed and the position thereof, on the basis of a missing region B in the displayed 3D model A.

As described above, with this embodiment, the current status of examination is recognized on the basis of the images during the endoscopy, and execution and stopping of the detection of unobserved regions are automatically switched between according to the status of examination. For example, when a doctor is identifying or treating a lesion site, the detection of unobserved regions is automatically stopped on the basis of conditions preset by the doctor. With this configuration, it is possible to prevent more unobserved regions than necessary from being detected for a user such as a doctor.

In addition, with this embodiment, it is possible to prevent the unobserved-region detection results from being excessively presented to the user during or after the endoscopy. For example, in the colonoscopy, there are cases where presentation of unobserved regions during the identification and treatment is unnecessary for the user. It is possible to prevent such unnecessary unobserved regions from being presented.

In addition, with this embodiment, processing algorithms used in the generation of a 3D model A and the detection of an unobserved region are determined according to the status of examination. This configuration makes it possible to generate a 3D model A and detect an unobserved region with higher precision.

In this embodiment, in a case in which one of identification and treatment is recognized, the processing determination unit 12 determines the processing mode on the basis of preset conditions; however, alternatively, the processing mode may be determined to be the stop mode.

Because the user is minutely examining the observation target during the identification and treatment, the detection of unobserved regions is generally unnecessary for the user. Therefore, by stopping the generation of a 3D model A and the detection of an unobserved region in the identification or treatment, it is possible to prevent more unobserved regions than necessary from being detected or to prevent unobserved regions from being excessively presented to a doctor.

Although the status recognition unit 11 recognizes identification or treatment as the status of examination in this embodiment, alternatively, or in addition thereto, the status recognition unit 11 may recognize other statuses in which the detection of unobserved regions is unnecessary for the user. In a case in which the other statuses are recognized, the processing determination unit 12 may determine the processing mode to be the stop mode, or may determine the processing mode on the basis of preset conditions.

One example of the other statuses is retroflexion in which the rear side of the endoscope 20 is observed by bending a bending portion of the endoscope 20 at a large angle. For example, in the colonoscopy, there are cases where the user minutely examines a specific site of the large intestine by means of retroflexion. During the retroflexion, a portion of the endoscope 20 is reflected in the image E. The status recognition unit 11 may recognize the portion of the endoscope 20 in the image E, thereby recognizing that the current status of examination is retroflexion.

Another example of the other statuses is quick movement of the endoscope 20, for example, movement at a speed equal to or greater than a prescribed value. In general, the user slowly moves the endoscope during observation of the observation target, but quickly moves the endoscope 20 when not observing the observation target (for example, during insertion of the endoscope 20 into the large intestine). In this case, the status recognition unit 11 recognizes the moving speed of the observation site in the image.

### Third Embodiment

Next, an image processing device, an image processing method, an image processing program, and a storage medium according to a third embodiment of the present disclosure will be described.

This embodiment differs from the first embodiment in the method of recognizing the status of examination and the method of determining a processing mode. In this embodiment, a configuration different from that of the first embodiment will be described, and parts having the same configuration as those in the first embodiment will be assigned the same reference signs, and descriptions thereof will be omitted.

As with the first embodiment, the image processing device 10 according to this embodiment is applied to the endoscope system 100 and includes the processor 1, the storage unit 2, the memory 3, and the input/output unit 4.

The processor 1 has, as functional units, the status recognition unit 11, the processing determination unit 12, the model generation unit 13, the unobserved region detection unit 14, and the display control unit 15.

The status recognition unit 11 recognizes the current status of examination on the basis of the image E. Specifically, in this embodiment, the status recognition unit 11 recognizes a hardly observable region in the image E, and on the basis of the area of the hardly observable region, the current status of examination is recognized as a status in which a 3D model A can be generated from the image E, or a status in which a 3D model A cannot be generated from the image E.

The hardly observable region is a region in which observation of an observation target, such as the mucous membrane, is difficult due to the fact that the observation target is hidden or unclear. In a case in which the area of the hardly observable region in the image E is less than a prescribed threshold, the current status of examination is recognized as a status in which a 3D model A can be generated. Meanwhile, in a case in which the area of the hardly observable region in the image E is equal to or greater than the prescribed threshold, the current status of examination is recognized as a status in which a 3D model A cannot be generated.

One example of the hardly observable region is a region of an occlusion (object) that covers the observation target. Examples of the occlusion include a residue, foam, blood, a treatment tool, a hemostatic clip, and the endoscope 20. For example, the status recognition unit 11 detects an occlusion in the image by means of the AI that has learned images including various occlusion.

Another example of the hardly observable region is a region with a poor image condition and is, for example, red-out, halation, fuzziness, or a blurred region. In this case, the image is an inappropriate image the entirety of which is the hardly observable region. Red-out is a phenomenon in which the entire image appears red as a result of the distal end of the endoscope 20 coming too close to the mucous membrane. For example, the status recognition unit 11 determines whether or not the image is an inappropriate image by means of the AI that has learned inappropriate images.

In the case in which the area of the hardly observable region is less than the prescribed threshold, the processing determination unit 12 determines the processing mode to be the execution mode. In this case, the model generation unit 13 generates a 3D model A, and the unobserved region detection unit 14 detects an unobserved region B in the 3D model A. When reflecting the difficult-to-observe area in the coverage ratio, the area of the difficult-to-observe area may be subtracted from the area of the object to be observed obtained in the 3D model reconstructed from the endoscopic image, and the area of the object to be observed from the image acquired by CT or MRI may be obtained and the area of the object to be observed from the created image may be subtracted. Since the coverage rate is the ratio of the observation area (numerator) based on this area (denominator), this difficult-to-observe region area may be subtracted only from the molecule. If you assume a scene where the scope is not moved much by paying attention to the lesion such as treatment or differentiation, it becomes meaningless to judge the difficult-to-observe area in that area that has already been sufficiently observed and confirmed. The value of this “stop mode” is not so much from the perspective of coverage as it is from the viewpoint of usability, and there is value in reducing the hassle.

Meanwhile, in the case in which the area of the hardly observable region in the image E is equal to or greater than the prescribed threshold, the processing determination unit 12 determines the processing mode to be the stop mode. In a case in which the image E is determined to be an inappropriate image, the processing mode is always determined to be the stop mode. In the case in which the processing mode is determined to be the stop mode, the model generation unit 13 does not execute the generation of a 3D model A, and the unobserved region detection unit 14 does not execute the detection of an unobserved region B.

In the case in which the processing mode is determined to be the execution mode, the processing determination unit 12 may further determine, on the basis of the recognition result for the hardly observable region, a processing algorithm for the generation of a 3D model A.

In one example, in a case in which an inhibitor is detected, the processing determination unit 12 may determine, as the processing algorithm, mask processing for the occlusion region. The model generation unit 13 applies the mask processing to the occlusion region in the image E, and generates a 3D model A by using a region other than the region subjected to the mask processing. By doing so, a 3D model A of the observation target that does not include an occlusion is generated.

The display control unit 15 may generate a display 7 and a report 8, described in the first embodiment, and may cause the display 7 and the report 8 to be displayed on the display device 30 (see FIGS. 3A to 5B). Alternatively, the display control unit 15 may cause the 3D model A, described in the second embodiment, to be displayed on the display device 30 (see FIGS. 7A and 7B).

Next, an image processing method executed by the image processing device 10 will be described.

As with the first embodiment, the image processing method according to this embodiment includes steps S1 to S6.

FIG. 9 shows step S2 and step S3 in this embodiment.

In step S2, the status recognition unit 11 recognizes a hardly observable region in the image E, and on the basis of the area of the hardly observable region, the current status of examination is recognized as a status in which a 3D model A can be generated, or a status in which a 3D model A cannot be generated (step S24).

Next, in step S3, in the case in which the current status of examination is a status in which a 3D model A can be generated, the processing determination unit 12 determines the processing mode to be the execution mode (“YES” in step S35), and subsequently determines a processing algorithm (step S36). In this case, steps S4, S5, and S6 are subsequently executed.

Meanwhile, in the case in which the current status of examination is a status in which a 3D model A cannot be generated, the processing determination unit 12 determines the processing mode to be the stop mode (“NO” in step S35). In this case, steps S4 and S5 are not executed and step S6 is executed next.

As described above, with this embodiment, the current status of examination is recognized on the basis of the images during the endoscopy, and execution and stopping of the detection of unobserved regions are automatically switched between according to the status of examination. For example, when the area of a non-observable region in the image is less than the prescribed threshold and a 3D model A can be generated, the detection of unobserved regions is executed, and when the area of the non-observable region in the image is equal to or greater than the prescribed threshold and a 3D model A cannot be generated, the detection of unobserved regions is automatically stopped. A 3D model A generated from the image including a large non-observable region lacks in accuracy, and an unobserved region detected from such an inaccurate 3D model A is unnecessary for the user.

Therefore, with this embodiment, it is possible to prevent more unobserved regions than necessary from being detected for a user such as a doctor. In addition, the detection result for an unobserved region, which is detected from an inaccurate 3D model A and has low reliability, can be prevented from being presented to the user.

In addition, with this embodiment, a processing algorithm to be used in the generation of a 3D model A is determined according to the recognition result for the hardly observable region. This configuration makes it possible to generate a 3D model A with higher precision.

Although the embodiments of the present disclosure and the modifications thereof have been described above with reference to the drawings, the specific configuration of the present disclosure is not limited to the abovementioned embodiments and modifications, and various design changes can be made within a range that does not depart from the scope of the present disclosure. In addition, the components illustrated in the abovementioned embodiments and modifications can be combined as appropriate.

For example, at least two of the first, second, and third embodiments may be implemented in combination.

In addition, in the individual embodiments, the display control unit 15 may generate other displays concerning the detection of the unobserved region B, in addition to the abovementioned displays. For example, the display control unit 15 may generate a display indicating that an unobserved region B has been detected or a display indicating the position of the detected unobserved region B, and may cause these displays to be displayed on the display device 30 in real time.

### EXAMPLE

An image processing device for processing an image captured by an endoscope, the image processing device comprising:
a processor,
wherein the processor executes:
recognizing, on a basis of the image captured by the endoscope, a current status of observation of an observation target, performed by a user;
determining the processing mode for the image based on the observation situation;
depending on the determined processing mode, a three-dimensional model of the observation object is generated from the image;
depending on the determined processing mode, an unobserved area not observed by the endoscope based on the three-dimensional model is detected;
determining a processing mode for the detection of the unobserved region based on the observation situation;
generating the three-dimensional model and detects the unobserved region according to the determined processing mode; A display screen related to the detection of the unobserved region is generated, and the display screen is output to a display device together.

### REFERENCE SIGNS LIST

1 processor
2 storage unit (storage medium)
2a image processing program
20 endoscope
A three-dimensional model
B unobserved region
E image

What is claimed is:

1. An image processing device, the image processing device comprising:
one or more processors comprising hardware, wherein the one or more processors being configured to:
identify a status of observation of an observation target based on an image captured by an endoscope;
determine, based on the status of observation, a processing mode concerning detection of an unobserved region that has not been observed by the endoscope; and
perform generation of a three-dimensional model of the observation target from the image and detection of the unobserved region according to the determined processing mode.

2. The image processing device according to claim 1, wherein
the processing mode includes an execution mode for executing detection of the unobserved region and a stop mode for stopping detection of the unobserved region, and
the one or more processors being configured to:
execute the generation of the three-dimensional model and the detection of the unobserved region where the processing mode is determined to be the execution mode, and
stop the generation of the three-dimensional model and the detection of the unobserved region where the processing mode is determined to be the stop mode.

3. The image processing device according to claim 1, wherein the processing mode includes a plurality of execution modes in which the unobserved region is detected with a different sensitivity in each of the plurality of execution modes.

4. The image processing device according to claim 1, wherein the identifying the status of observation includes recognizing an observation site in the image.

5. The image processing device according to claim 1, wherein the identifying the status of observation includes identifying at least one of a lesion site, a treatment tool, an operation of a treatment tool, a staining solution, or special light, in the image, or movement of an observation site in the image.

6. The image processing device according to claim 1, wherein the identifying the status of observation includes identifying at least a part of the endoscope in the image.

7. The image processing device according to claim 1, wherein the identifying the status of observation includes identifying a moving speed of an observation site in the image.

8. The image processing device according to claim 1, wherein the identifying the status of observation includes determining whether the image is appropriate for generating the 3D model.

9. The image processing device according to claim 8, wherein the determining whether there the image is appropriate for generating the 3D model includes determining whether the image includes at least one of an object covering the observation target, a red-out region, a halation region, a fuzzy region, or a blur region.

10. An image processing method, the image processing method comprising:
identifying a status of observation of an observation target based on an image captured by an endoscope;
determining, based on the status of observation, a processing mode concerning detection of an unobserved region that has not been observed by the endoscope,
generating a three-dimensional model of the observation target from the image; and
detecting of the unobserved region are performed according to the processing mode.

11. The image processing method according to claim 10, wherein
the processing mode includes an execution mode for executing detection of the unobserved region and a stop mode for stopping detection of the unobserved region, and
the one or more processors being configured to:
execute the generation of the three-dimensional model and the detection of the unobserved region where the processing mode is determined to be the execution mode, and
stop the generation of the three-dimensional model and the detection of the unobserved region where the processing mode is determined to be the stop mode.

12. The image processing method according to claim 10, wherein the processing mode includes a plurality of execution modes in which the unobserved region is detected with a different sensitivity in each of the plurality of execution modes.

13. The image processing method according to claim 10, wherein the identifying the status of observation includes recognizing an observation site in the image.

14. The image processing method according to claim 10, wherein the identifying the status of observation includes identifying at least one of a lesion site, a treatment tool, an operation of a treatment tool, a staining solution, or special light, in the image, or movement of an observation site in the image.

15. The image processing method according to claim 10, wherein the identifying the status of observation includes identifying at least a part of the endoscope in the image.

16. The image processing method according to claim 10, wherein the identifying the status of observation includes identifying a moving speed of an observation site in the image.

17. The image processing method according to claim 10, wherein the identifying the status of observation includes determining whether the image is appropriate for generating the 3D model.

18. The image processing method according to claim 17, wherein the determining whether there the image is appropriate for generating the 3D model includes determining whether the image includes at least one of an object covering the observation target, a red-out region, a halation region, a fuzzy region, or a blur region.

19. A non-transitory computer-readable storage medium that stores an image processing program, wherein the image processing program causes the computer to execute:
identifying a status of observation of an observation target based on an image captured by an endoscope;
determining, based on the status of observation, a processing mode concerning detection of an unobserved region that has not been observed by the endoscope; and
generating a three-dimensional model of the observation target from the image and detecting of the unobserved region according to the processing mode.
